(19) United States
(12) Patent Application Publication
(10) Pub. No.: US 2008/0261902 A1
(43) Pub. Date: Oct. 23, 2008

(54) Pharmaceutical composition containing polydatin and its application

(75) Inventors: Jinhua Zhao, Nanshan Shenzhen (CN); Hui Kang, Nanshan Shenzhen (CN); Weizhen Zeng, Nanshan Shenzhen (CN); Lijuan Zhang, Nanshan Shenzhen (CN); Dan Zhu, Nanshan Shenzhen (CN); Chuangui Huang, Nanshan Shenzhen (CN); Hong Chen, Nanshan Shenzhen (CN); Hanlin Feng, Nanshan Shenzhen (CN); Lin Yu, Nanshan Shenzhen (CN); Xuliang Huang, Nanshan Shenzhen (CN); Guiling Zhao, Nanshan Shenzhen (CN); Kesen Zhao, Nanshan Shenzhen (CN)

Correspondence Address: DAVID AND RAYMOND PATENT FIRM, 108 N. YNEZ AVE., SUITE 128, MONTEREY PARK, CA, 91754, US

(73) Assignee: Shenzhen Neptunus Pharmaceutical Co., Ltd.

(21) Appl. No.: 12/157,810

(22) Filed: Jun. 13, 2008

Related U.S. Application Data
(63) Continuation-in-part of application No. 10/492,405, filed on Apr. 4, 2005.

(51) Int. Cl. A61K 31/7034; A61P 7/04

(52) U.S. Cl. 514/35

(57) ABSTRACT

A composition includes polydatin and solvent for dissolving the polydatin, wherein the solvent comprises alcohol in a range between 40 and 95% by volume, propanediol in a range between 0 and 60% by volume, and water in remaining volume. The concentration of the polydatin must be at least 6 mg/ml. The composition has good stability in cold preservation. The composition is in a pharmaceutically acceptable administrative form selected from the group consisting of an intravenous injection, and intramuscular injection, an oral administration, a spray solution, and an aerosol form.

### CROSS REFERENCE OF RELATED APPLICATION

This is a CIP application of a non-provisional application having an application Ser. No. 10/492,405 and a filing date of Apr. 04, 2005.

### BACKGROUND OF THE PRESENT INVENTION

1. Field of Invention

The present invention relates to a pharmaceutical composition containing polydatin and application thereof.

2. Description of Related Arts

Physiology researches show that the physiological function of the microcirculation mainly includes transporting oxygen, nutrients, physiological regulating active substances to cells, and removing metabolic wastes so as to maintain the normal activities of life. Many kinds of diseases are related to or arisen from the abnormal microcirculation, such as acute inflammatory diseases, traumatism, shock, chronic ulcer, cirrhosis, cardiovascular diseases, and diabetes mellitus. Therefore, improving the microcirculation is a very important and meaningful subject in the treatment and prevention of many diseases, and it is a highly recognizable subject in the medical and pharmaceutical industry for finding an effective medication for improving microcirculation.

Anisodamine hydrobromide, Atropine and Anisodine are the exemplary conventional medications for improving the microcirculation system, which share the common characteristics of having the effect of dilation of capillary, increasing the blood flow of tissues and organs. They are widely used in the treatment of different diseases.

Polydatin was first found in the plant Polygonum cuspidatum Sieb. Et Zucc. in the 60's in the 20 century (Nomura, 1963), and was then found in other plants such as peicin, grape and peanut. The chemical formula of polydatin is 3, 4′, 5-trihydroxy-trans-stilbene-3-b-d-glucose with the 3, 4′, 5-trihydroxy, also called resveratrol, as glucoside, is a stilbene compound. The chemical structure of polydatin is shown as follows:

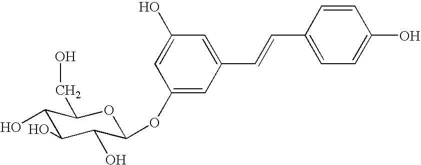

Since the 70's of the 20 century, a variety of researches of the pharmaceutical effect and activities of polydatin have been conducted in China. It is found that polydatin has the effect of lowering the cholesterol level of blood, that the use of 2.2 mg/kg/d polydatin is capable of lowering the total cholesterol, LDL/HDL cholesterol ratio, and blood platelet aggregation rate of a hyperlipoidemia patient (ZHANG, Pei Wen, 1995), the effect of inhibiting the aggregation of blood platelet and the formation of thrombus, that 6˜100 μmol/L polydatin is capable of inhibiting the production of the platelet accumulation and B₂ thrombus which are induced by AA and ADP (SHAN, Chun Wen, 1993), the effect of inhibiting blood platelet aggregation induced by adrenaline (YANG, Su Qin, 1992), the effect of inhibiting the blood platelet aggregation and the formation of thrombus caused by damage in common carotid artery in rabbit (by WANG, Yue Zhong, 1995), the effect of inhibiting the deformation and release reaction of blood platelet (by LIU, Lian Pu, 1998), the effect of inhibiting adhesive nature of white blood cells on inner wall of cell (by JIN, Chun Hua, 1998), the effect of strengthen the muscular strength (JIN, Xing Zhong, LUO, Su Fang, 1992), the effect of strengthen myocardial contraction and frequency (JIN, Chun Hua, 2000), the effect of vascular dilation such as the effect of promoting separated pulmonary artery dilation (LUO, Su Fang, 1992), the effect of contraction of main artery induced by non-competitive restrained platelet (WANG, Yue Zhong, 1994), the effect of anti-oxidation such as the inhibiting or removing function of polydatin on free radicals produced by PMNs of blood of human, Xanthin-Xanthin system, and VitC-Cu²⁺ system (JIN, Wei Jun, 1993), the effect of reducing damages to intestine and organs of rabbit (XIE, Zhong Lin, 1997), the effect of improving microcirculation and treatment of shock such as having treatment effect to shock caused by bleeding in rats which is better than that of using an equal dose of Dopamine and Anisodamine hydrobromide (HUANG, Qiao Bing, 1994), the effect of preventing and treating microcirculation of rats caused by burning (by WU, Kun Ying, 1992), the effect of reducing blood pressure difference of capillaries caused by bleeding and restoring the flowing of blood in capillaries (ZHU, Zuo Jiang, 1989), the effect of protecting damages to lungs caused by internal intoxication in rats (MO, Guo Yu, 1993), the effect of inhibiting increase in endothelin concentration of external blood flow induced by damages and decreasing the level of carbon monoxide (ZHAO, Wu Min, 1998), the effect of restoring and regulating body activities for burnt (XU, Chuan Lin, 1994). Furthermore, polydatin has liver protective function (HUANG, Zhao Sheng, 1998), and regulatory function in immune system (LUO, Rong Jiang, 1992).

Recently, many researches on the glucoside of polydatin, that is resveratrol, have been carried out and attracted international attention. ‘France paradox’ has been analyzed in 1992 and the cardiovascular protective function of resvertrol has been reported. Then, more and more documentations reported the pharmaceutical activity of resvertrol, such as, report of the anti-oxidative function and inhibiting effect on LDL peroxidation for preventing arteoclerosis (Pace-Asciak, 1996), and report on inhibiting the formation and developing of tumor (Jang M, Udeani GO et, Science 1997; 275: 218).

Recently, patent related documentations can also be found. For example, the PCT application WO 01/30336 A2 has disclosed the use of resvertrol for treating epidermal diseases, the PCT application WO 01/91714 A1 has disclosed the use of resvertrol in cosmetic products for treating dandruff or scurf, the PCT application WO 00/38620 has disclosed the use of resvertrol on treating periodontal diseases, the PCT application WO 00/12534 has disclosed the use of stilbene composition in treating hepatitis, the European patent EP 1138323 A2 has disclosed the use of resvertrol on treating eczema and psoriasis, the U.S. Pat. No. 6,008,260 A has disclosed the use of resvertrol on preventing tumor, the PCT application WO 00/44370 has disclosed the use of resvertrol derivative on treating osteoporosis and high blood pressure, the China patent 00121100.5 has disclosed the extraction of resvertrol and polydatin, the China patent 99115156.9 has disclosed the extraction of resvertrol from polydatin, the China patent 00113914.2 has disclosed the production method of cell having the composition resvertrol.

However, reports of composition of polydatin or its derivatives for improving microcirculation and treatment or preventing shock symptoms are not found. Moreover, the water solubility of polydatin is relatively poor which poses difficulties in the technical applications of polydatin in making composition for venous injection. Therefore, the polydatin used for venous injection is not found in different documentation in patent or patent applications.

In 1963, Nomura Sinta separated polydatin from Polygonum cuspidatum Sieb. Et Zucc., and found that the polydatin also exits in spruce of sink leaves, grape, peanut and etc. Polydatin has structure of 3,4′,5-trihydroxy-trans stilbene-3-β-D-glucoside, is trans stilbene, and the structural formula is as below.

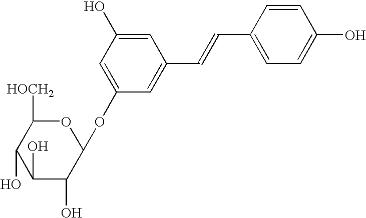

Researches of the recent tens of years indicate that the polydatin has physiological activity of improving microcirculation, reducing blood lipid and resisting tumor. However, there is not clinic preparation of the compound on sale.

In basic pharmacological researches, and as for in vivo experiments, administration of the polydatin adopts ways in vein mostly, and experimental sample adopts water or normal saline to prepare polydatin pharmaceutical composition of concentration of 2˜5 mg/ml mostly. Nonetheless, there is not further description about feasible preparation methods of the solution in prior documents. The following lists include administration dose and experimental drug concentration of the polydatin according to partial pharmacological research documents.
1. Wang Lijing, etc., Experimental treatment of multiple organ failure after crush injury with polydatin in rats, CHINESE JOURNAL OF PATHOPHYSIOLOGY, 1995, 11(1): 70˜73: Administration dose is 30 mg/kg, and Concentration of solution is 3 mg/ml.
2. Jiangyong, etc., THE CHANGE IN FLOW DISTRIBUTION AND THE EFFECT OF POLYGONUM CUSPID ATUM ON RAT MICROVASCULATURE DURING HEMORRHAGIC SHOCK, Medical Journal of Chinese People's Liberation Army, 1993, 18(6): 405˜408: Administration dose is 10 mg/kg, and Concentration of solution is 5 mg/ml.
3. Zhu zuojiang, etc., Effect of crystal NO_{—}4 of polygonum cuspidatum on pulse pressure difference and microcirculation perfusion flow in rat during shock, National Medical Journal of China, 1989, 69(5): 279˜281: Administration dose is 10 mg/kg, and Concentration of solution is 3.3 mg/ml.
4. Zhu zuojiang, etc., Effect of crystal NO_{—}4 of polygonum cuspidatum on the left ventricular contractile function in rabbit during hemorrhagic shock, Chinese Journal of Pathophysiology, 1991, 7(4): 349˜351: Administration dose is 3.3 mg/kg, and Concentration of solution is 5.5 mg/ml.
5. Zhao kesen, etc., The prognostic implication of determination of activation of leucocyte in severe burns, CHINESE JOURNAL OF PLASTIC SURGERY AND BURNS 1989, 9(4):279˜282: Administration dose is 10 mg/kg, and Concentration of solution is 5.5 mg/ml.
6. Wu kunying, etc., Changes of Plasma Tumor Necrosis Factor Level and Microcirculation in Rats During Burn Shock, CHINESE JOURNAL OF MICROCIRCULATION, 1995, 5(4): 3˜5: Administration dose is 10 mg/kg, and Concentration of solution is 5 mg/ml.
7. Wang yanchun, The prognostic implication of determination of activation of leucocyte in severe burns, CHINESE JOURNAL OF PLASTIC SURGERY AND BURNS, 1994, 10(7):286: Administration dose is 10 mg/kg, and Concentration of solution is 5 mg/ml.
8. Wu kunying, etc., Relationship between disturbances of microcirculation and TNF during burn shock, CHINESE JOURNAL OF PLASTIC SURGERY AND BURNS, 1996, 12(1): 41˜44: Administration dose is 10 mg/kg, and Concentration of solution is 5 mg/ml (0.5%) diluted with NS.
9. Wu kunying, etc., effect of NO_{—}4 of polygonum cuspidatum on the microcirculation disorder of rats, CHINESE JOURNAL OF PLASTIC SURGERY AND BURNS, 1994, 10(7):286, 1992, 8(2): 133: Administration dose is 10 mg/kg, and Concentration of solution is 5 mg/ml.
10. Zhao kesen, etc., effect of NO_{—}4 of polygonum cuspidatum on increasing early heart function of burning shock, CHINESE JOURNAL OF PLASTIC SURGERY AND BURNS, 1994, 10(7):286, 1992, 8(2): 133, 1989, (4): 275˜278: Administration dose is 10 mg/kg, and Concentration of solution is 5 mg/ml (0.5%).
11. Zhangman, etc., The effect of polydatin on endotoxic shock in rats, Chinese Journal of Pathophysiology, 1997,13(3):245˜248: Administration dose is 10 mg/kg, and Concentration of solution is 5 mg/ml (0.5%).
12. Mo guoyu, etc., Protective effect of polydatin on endotoxic lung injury in rats, Chinese Journal of Tuberculosis and Respiratory Diseases, 1993, 6(3): 153˜154: Administration dose is 10 mg/kg, and Concentration of solution is 2 mg/ml NS solution.
13. Zhangman, etc., Protective effect of polydatin on multiple organ injury in rats with endotoxic shock, CHINESE CRITICAL CARE MEDICINE, 1995,7(6): 352˜354: Administration dose is 10 mg/kg, and Concentration of solution is 5 mg/ml (0.5%).
14. Zhang mingjing, etc., The effect of polydatin (PD) on the imbalance of Th1/Th2 type cytokines with endotoxic shock-induced lung injury in rats, Journal of Pediatric Pharmacy, 2003,9(3): 1˜4: Administration dose is 10 mg/kg, and Concentration of solution is 5 mg/ml (0.5%).
15. Shu shiyu, etc., The effect of polydatin (PD) on the endotoxic shock-induced lung injury in rats, Journal of Applied Clinical Pediatrics, 2002, 17: 121: Administration dose is 10 mg/kg, and Concentration of solution is 5 mg/ml (0.5%).
16. Wang xingyong, etc., The in terfered effects of polydatin in endotoxic shock-induced lung injury, Modern Medicine Health, 2003, 19(3): 256˜258: Administration dose is 10 mg/kg, and Concentration of solution is 5 mg/ml (0.5%).
17. Xie zhonglin, etc., Polydatin prevents remote organ damage during intestinal ischemia reperfusion, CHINESE JOURNAL OF PATHOPHYSIOLOGY, 1995, 11(5): 528·531: Administration dose is 2.5 mg/kg, and Concentration of solution is 2 mg/ml (0.2%).
18. Wangyu, etc., study on resisting forming of thrombus and improving microcirculation of polydatin, Shaanxi Journal of Traditional Chinese Medicine, 2003, 24(7): 663˜665: Administration dose is 25, 50, 100 mg/kg/d, and Concentration of solution is distilled water, suspension.
19. Jin xingzhong, etc., Effect of polydatin on the myocardial functions in the isolated working rat heart, Journal of First Military Medical University, 1992, 12(1): 1˜33: Administration dose is 0.11 mmol/L, and Concentration of solution is dissolved by heating, 3.4 mg/ml (0.34%).

On the other hand, according to the prior pharmacological documents, the clinic effective dose can be predicted. For example, in various experiments using rats as experimental animal, single administration dose of the polydatin is 10 mg/kg body weight or more; and according to the body surface area calculation method adopted standard in the present field, dose used to human beings (calculated by 70 kg) should be above 112 mg/time with respect to the doses of rats. Obviously, as adopting normal clinic injection specification of 1˜20 ml, the polydatin concentration in the solution should not be lower than 5.6 mg/ml.

Actually, the polydatin has limited solubility in normal water or normal saline, whose conventional water solution hardly reaches the concentration described in the above documents. For example, the polydatin has saturated concentration lower than 0.5 mg/ml in water and normal saline under room temperature, which is much lower than the described administration concentration of 2˜5 mg/ml.

Adjusting pH, heating, adding surfactants, and etc., are conventional methods of increasing solubility of compounds, but researches indicate that the methods have limited significance as preparing stable polydatin drug compositions of effective concentration.

For example, adjusting pH of solution can increase solubility of polydatin, but to obtain polydatin water solution having concentration above 3 mg/ml, the solution will have pH above 12 approximately, and the final solution will have pH above 10 approximately. It is widely known by technicians in the field that clinic solutions require pH4.0˜9.0. Obviously, it is hard to prepare polydatin water solution of effective concentration by adjusting pH only. Furthermore, the polydatin has bad stability in solutions of high pH, so the clinic application of the polydatin drug composition preparations of high pH will be further limited.

The described document No. 19 adopts heating to dissolve to obtain polydatin drug compositions. According to the experiment, the solutions obtained by heating to dissolve precipitate solute as the temperature reduces, and the polydatin concentrations in the solutions are lower than 0.5 mg/ml after indoor temperature balancing. Obviously, the method is not suitable in preparing officinal medicine preparations.

China Patent Application 02134928.2 describes a method of preparing polydatin solutions for injection by adopting adjusting pH and/or adding surfactants (tween 80). The research indicates that as preparing a polydatin drug composition containing tween 80 of 0.5% and of pH8.5, when the concentration of the solution reaches 4 mg/ml, the solution can not be cold preserved (polydatin crystal is precipitated in cold preservation of 4° C.). Due to the fact that the polydatin (solution) is sensitive to temperature and light, low temperature and avoiding light is preferable storage condition for polydatin preparations (solution), and cold preservation is conventional storage condition for keeping the active component of injection stable, so, injections unsuitable to store in the described conditions have defects in medical storage. Furthermore, the prior documents indicate that preparations of administration in vein should be avoided to use as possible because tween surfactants have potential hemolytic and antihypertensive activity. (Zhuangyue etc., applied drug preparation technique, People's medical publishing house 1999:p417)

Utilizing propanediol as latent solvent can increase solubility of the polydatin. Chinese patent application 02134928.2 describes a method of preparing solutions for injection with 5%˜20% propanediol, however, researches indicate that the polydatin has solubility lower than 5 mg/ml in 20% propanediol water solution, about 3.5 mg/ml, and the solution is not stable, which precipitates solute of polydatin after 12 hours. Besides, the polydatin solution containing 20% propanediol, 0.5% tween, of pH8.5, and having concentration of 8 mg/ml prepared according to methods of embodiment 1-2 of the China patent 02134928.2, precipitates polydatin crystal after long cold preservation, too; diluting the unit preparation according to the embodiment with 100 ml 0.9% sodium chloride injection (normal saline), the compatible solution obtained is not stable, and precipitates crystal.

Obviously, in range allowed by clinic preparation, adjusting pH, adding tween surfactants, or merely utilizing propanediol as latent solvent can not obtain satisfactory preparation schemes of clinic polydatin injection. As a result, studying and developing stable drug composition of solution containing polydatin of effective concentration has great practical significance to realize clinic application of the polydatin.

### SUMMARY OF THE PRESENT INVENTION

A main object of the present invention is to provide a pharmaceutical composition having a quantity of polydatin or a quantity of salt of polydatin for improving the microcirculation. The pharmaceutical composition is capable of treating or preventing related obstructive diseases of microcirculation, wherein the composition comprising a quantity of polydatin or salt of polydatin is administered into a user.

Another object of the present invention is to provide a method of making a composition of polydatin or its salt for application on making a composition for improving microcirculation.

Accordingly, in order to accomplish the above objects, the present invention comprises a pharmaceutical composition capable of treating or preventing obstructive microcirculation related diseases, wherein the composition comprises an active composition of polydatin or a salt of polydatin, and one or more than one vehicle. The chemical formula of polydatin is 3, 4′, 5-trihydroxy-trans-stilbene-3-β-D-glucose.

The salt of polydatin is defined as a salt or an ester formed by polydatin and a predetermined acid including an organic acid or an inorganic acid, or and a predetermined alkaline including an organic or an inorganic alkaline.

One of the embodiments of the present invention is to provide a composition comprising polydatin or its salts which is applied externally, wherein the composition which is applied externally may be in the forms of solution, or a predetermined carrier such as injection solution, injection powder adapted for making an aqueous solution, or a frozen dry injection powder.

A preferred solvent of the aqueous solution of the present invention is physiological buffer or physiological saline which is applicable to a body. When making the aqueous solution for injection, the pH of the aqueous solution may be suitably adjusted such that the aqueous solution is neutral or alkaline under acceptable pharmaceutical conditions. For example, a range between pH 7.0 and pH 10.0 is capable of increasing the solubility of the active composition. The pH is preferably adjusted by using sodium carbonate (Na₂CO₃), sodium bicarbonate (NaHCO₃), or sodium hydroxide (NaOH) to adjust to pH 8.0 to 9.5, or by using a PBS buffer having a pH range between 8.0 and 9.5. In one of the embodiment of the present invention, the aqueous solution comprises 10 to 30% propandiol and 30 to 50% ethanol, and that the aqueous solution is diluted 25 to 50 times which is applicable to venous, muscular, or intravenous injection.

The aqueous solution of the present invention comprises a predetermined solubilizing agent, a lacquer solvent or a cosolvent for increasing the stability and solubility of the active composition of the present invention. For example, the aqueous solution comprises a predetermined quantity of low alcohol such as 5 to 20% 1, 2-propandiol which is capable of increasing the solubility of the active composition of the present invention, and a predetermined quantity of superficial active agent such as 0.1 to 1% Tween 80 which is capable of increasing the stability of the aqueous solution. The solvent may further also be a cosolvent system comprising propandiol, manitol, non-polar superficial active agent, hydrated organic polymer, and cosolvent system in water phase.

A process of making the cool dry injection powder of the present invention comprises the step of providing an aqueous solution of the active composition of the present invention and cooling and drying under a suitable cool dry condition.

A composition of the present invention is capable of making a suspension solution by mixing lipophilic solvent or carrier and the active composition of the present invention. Examples of the lipophilic solvent or carrier include fatty oil or fatty acid ester such as triacylglycerol or liposome. The suspended solution may further comprise composition which increases the adhesion level such as sodium carboxyl methyl cellulose, sorbitol, or glucosans. A predetermined stabilizing agent or composition increasing solubility may be added for making concentrated aqueous solution.

The composition for external application preferably comprises the active composition having unit dose wherein the unit dose may comprise polydatin or its salt between the range of 1.0-1000 mg and the preferred unit dose is between 50 mg and 250 mg.

A second embodiment of the present invention is providing an oral application of the polydatin or its salts which is pharmaceutical acceptable.

The composition of the present invention for oral application may be in the form of tablet, pill, granule, capsule, solution, gel, syrup, and suspended solution.

A method of making the composition of the present invention for oral application may comprise a step of adding a predetermined quantity of vehicle such as microcrystallized cellulose, sucrose, lactose, glucose, starch, and manitol, binding agent such as syrup, Arabic gum, silica gel, sorbitol, tragacanth gum, methyl cellulose and polyethylene pyrrolidone; adding a predetermined quantity of disintegrating agent such as starch, carboxyl methyl cellulose and its calcium salt, microcrystalline cellulose, polyethylene glycol, cross-link polyethylene pyrrolidone, bubbler, alginic acid or its salt such as sodium alginate; adding a predetermined quantity of glidant such as talc powder, magnesium stearate, calcium stearate, and silica, lubricating agent such as magnesium stearate, sodium luarate, and glycerol, or the like. The method may also comprise the steps of mixing the active composition and a solid vehicle and grounding the mixed composition and vehicle to form a mixture, adding a predetermined auxiliary substance if necessary, and making the mixture into the form of a granule. The method may further comprise the steps of transforming the mixture in the form of a granule into the form of tablet or substance for coating with sugar, and the substance for coating with sugar is then properly coated by a coating materials such as concentrated sugar solution selected from the group comprising Arabia gum, talc powder, polyethylene pyrrolidone, resin of carboxyl polymer, polyethylene glycol, and/or titanium dioxide, lacquer solution, and a suitable organic solvent or solvent mixture. The mixture in the forms of tablet or pill may combine with a dye or color substance for distinguishing or representing a different dosage group.

The forms of oral administering composition further include capsule form made with gelatin, and soft capsule made from gelatin and plasticizing agent. The capsule form may comprise active composition and supplementary agent such as lactose, binding agent such as starch and/or lubricating agent such as talc or magnesium stearate, and stabilizing agent. The soft capsule form may comprise soluble active composition or suspension in suitable solution such as fatty oil or aqueous polyethylene glycol. Furthermore, stabilizing agent or lacquer solvent.

The oral administrative composition of the present invention is preferred to have a predetermined active composition in the quantity of unit dose, the unit dose can be polydatin or its salt in the range of 1.0 to 1000 mg, and preferably of 50 to 250 mg.

The composition of the present may also be in the form a rectal suppository comprising polydatin or its salt in the range of 1.0 to 1000 mg and a suppository agent such as coconut oil or other glyceride.

The composition of the present invention may also be localized administrative agent such as paste, cream, sticker, coating agent form comprising polydatin or its salt in the range of 1.0 to 1000 mg, and common supplementary substances such as Vaseline, glycerol, sodium lauryl sulfate, mono stearo glyceride, acetyl cellulose, bubblering oil, paraffin oil, and lanonine.

The method of administration of the composition of the present invention includes oral administration, eye drops administration, rectal suppository, membrane permeable administration, localized administration, or intestinal administration; external administration such as muscular, intravenous, marrow injection, and intrathecal or direct internal injection, venous abdominal or intraocular injection.

The composition of the present invention has significant dilation effect on capillaries, lowering the blood viscosity level of capillaries, inhibiting the blood cell aggregation, increasing the microcapillaries dilation rate, and hence has significant effect on improving the microcirculation and is applicable in treatment or preventive measure of microcirculation obstructive disease.

The composition of the present invention can also be used for preventing or treating shock.

One of the symptoms of shock is microcirculation obstruction which induces insufficient irrigation or circulation of blood to important organs and may result in deterioration of function of organs. The active composition of the composition of the present invention is capable of exerting dilating effect to the microvascular vessels of animals, increasing the opening rate of capillaries of organs, increasing difference in blood pressure, thus is capable of effectively improving the blood circulation for different organs under shocking conditions and increasing the animal survival number in the shock model experiment.

In the treatment for shock, venous administration is preferred such as venous injection or instillation. The dosage of the composition depends on the body weight, age, body conditions and is decided by a practitioner. For an adult, a preferred suitable dose is about 20 to 600 mg per dose, preferably 80 to 300 mg per dose.

The present invention is capable of using for treating or preventing cardiovascular and cerebral vascular diseases.

Cardiovascular and cerebral vascular diseases are usually related to microcirculation obstruction. For example, cerebral hypoxia, insufficient blood flow resulted from insufficient supply of blood. The active composition of the composition of the present invention can improve the microcirculation and thus improve the blood flow to heart and brain such that the aim for treating or preventing cardiovascular and cerebral vascular diseases is achieved.

When the composition of the present invention is used for treating or preventing vascular diseases, the composition can be in the form of oral administrable medication or medication for external use. The appropriate dosage is decided by practitioner according to the weight, age, body conditions of the patient. The appropriate dosage for an adult is 20-600 mg per day which can be applied in a single dose or divided into several doses. The preferable dose is 20-80 mg per application, at a frequency of 2-3 doses for day.

The present invention is capable of using as a medication for treatment and preventive measure for some of the obstructive diseases of sense organs.

Some of the obstructive diseases of sense organs such as visual acuity decreases from microcirculation obstruction, or deaf caused by microcirculation obstruction. The active composition of the pharmaceutical composition of the present invention can improve the circulation of these sense organs and thus treating and preventing the visual and hearing obstructive diseases.

When the composition is used for treating or preventing visual and hearing diseases in eye and ear, the applications of the composition of the present invention can be applied as system administration such as oral administration or venous administration, and as localized administration such as eye dropping and intraocular injection. The appropriate dosage is decided by practitioner according to the weight, age, body condition of the patient. In the application as system administration, the appropriate daily dosage is between 20 and 600 mg which can be applied as a single dose or divided into separated dose.

The composition of the present invention can also be used for treating or preventing other related diseases of microcirculation obstruction. For example, the composition of the present invention can be used for venous thrombosis, external hemorrhoid caused by blood clot, skin damage or burn. The application of treating and preventing these diseases by the composition of the present invention can be system administration or localized administration.

The present invention is first using polydatin composition for improving the microcirculation in the pharmaceutical mediation, such that can be used for treating and preventing a variety of microcirculation obstructive diseases. In those intravenous medications, since the solubility of polydatin is relatively poor, under normal solubility condition and medication volume, it is difficult to provide an effective quantity of active composition in its aqueous solution. The present invention comprises alkaline solvent and solubilizing agent for effectively increasing the solubility of polydatin.

Another main object of the present invention is to provide a pharmaceutical composition containing polydatin and its application thereof, wherein the pharmaceutical composition comprises polydatin of effective concentration, and is capable of keeping stable as being cold preserved. The composition is prepared to compatible solution according to clinic conventional usage and dose, and is capable of keeping the compatible solution stable and clarified.

In the disclosed documents, the extraction of the polydatin from Polygonum cuspidatum Sieb. Et Zucc. with solvent of alcohol has been reported. Application of the alcohol in separating and purifying the polydatin has been reported, too. However, application of the alcohol in medicinal preparation containing the polydatin has not been reported. More importantly, dissolution behavior characteristics of the polydatin in the alcohol and mixed solvents containing the alcohol have not been reported. The documents and researches indicate that the polydatin has good solubility in the alcohol. On the other hand, the documents and researches also indicate that the polydatin has very low solubility in pure water (<0.5 mg/ml) under room temperature (25° C.). Generally, after a solute dissolves in good solvent, adding a solvent of low solubility will reduce the solubility of the solute in the good solvent evidently. The present field adopts “water extracting-alcohol precipitating” or “alcohol extracting-water precipitating” to separate extractions or utilizes mixed solvents to recrystallize basing on the principle. However, by researches the inventors discover that the polydatin has special dissolution behavior in an alcohol-water mixed solvent, which has a totally different principle from the described general principle. As mixing the alcohol, which is a good solvent for the polydatin, with the water, which is a solvent of low solubility for the polydatin, within a certain proportion range, when the bad solvent increases, the solubility of the polydatin in the mixed solution does not decrease. However, it increases surprisingly. For example, the polydatin has the solubility <0.5 mg/ml in pure water under room temperature; solubility about 20 mg/ml in anhydrous alcohol. Surprisingly, in alcohol water solution about 50%˜95%(v/v), the polydatin has solubility over 20 mg/ml. For example, in the 50% alcohol water solution, the polydatin has the solubility about 25 mg/ml, and in the 90% alcohol water solution, the polydatin has the solubility about 75 mg/ml. The result indicates that the alcohol water solution of 50%˜95%(v/v) has co-solvent effect, namely, within the certain proportion range, the solubility of the polydatin in the alcohol-water mixed solvent is better than any single solvent of the pure water or the pure alcohol. The researches also discover that in 15% alcohol-water solution, the polydatin has solubility about 1 mg/ml; in 30% alcohol-water solution, the polydatin has solubility about 6 mg/ml; namely, the co-solvent effect of the mixed solvent will no longer exit as exceeding the certain proportion range.

The inventors further discover by chance that, if adding a certain amount of propanediol to make a new mixed solvent in the described alcohol-water solution, the solubility of the polydatin in the mixed solvent will further increase in a certain proportion range, namely, is higher than solubility in any corresponding single solvent (alcohol-water, propanediol). For example, making a new mixed solvent with 60% alcohol-water and propanediol in the proportion of 10:25(v/v), the polydatin has solubility about 75 mg/ml, much higher than solubility in the 60% alcohol-water and the solvent of propanediol (about 42 mg/ml and 60 mg/ml respectively). As observing in cold preservation, the solution has good stabilization. The researches also discover that, the co-solvent effect of the mixed solvent will no longer exit as exceeding the certain proportion range.

As for administration in vein, concentration of alcohol in injection cannot be too high. Due to the fact, the present invention adopts a dense solution for injection to realize injections of continuous intravenous infusion administration. The dense solution for injection is an injection which is diluted to predetermined times with normal saline for injection as about to use, then administrated by way of intravenous bolus or intravenous infusion. Experiments indicate that a polydatin injection prepared with alcohol-water or water-alcohol-propanediol mixed solvent has good compatible stability in normal saline for injection, while a polydatin injection prepared with propanediol-water does not have stable compatibility. Besides, the researches indicate that in some certain condition, the mixed solvent has better clinic compatible stability than alcohol-water, and the polydatin injection has different compatible stability according to the mixed solvent of different preparation proportion.

Therefore, the present invention clarifies for the first time that the alcohol-water and/or propanediol mixed solvent of proper proportion can provide particular co-solvent effect to the polydatin; and only the alcohol-water and/or propanediol mixed solvent of proper proportion can prepare the dense solution for injection of compatible stability, and the solution can be cold preserved for long. The above properties have great significance in preparing medicinal preparation capable of applying in clinic.

Because water, alcohol, and propanediol are acceptable solvents in pharmacy, according to the object of the present invention and the above discovery, the present invention provides a drug composition of solution containing polydatin of high concentration. In the present application, the drug composition containing polydatin is called polydatin drug composition for short sometimes.

The polydatin pharmaceutical composition according to the present invention, comprises polydatin, and solvent for dissolving the polydatin, wherein calculated by volume, the solvent comprises 40%˜95% alcohol, 0%˜60% propanediol, and water of remaining amount.

In the present application, the described percentage content is volume percentage content without particular specification. According to the Chinese Pharmacopoeia, solvent alcohol is 95% alcohol without particular specification.

In the polydatin drug composition according to the present invention, the polydatin has concentration not lower than 6 mg/ml in the solvent generally.

Further, the polydatin has concentration of 6 mg˜150 mg/ml in the solvent.

Preferably, the polydatin has concentration of 20 mg˜100 mg/ml in the solvent.

In the polydatin pharmaceutical composition according to the present invention, the polydatin should have concentration no less than 6 mg/ml, because clinic effective dose can be predicted according to prior pharmacological research documents. For example, in various experiments using rats as experimental animal, single administration dose of the polydatin is 10 mg/kg body weight or more; and according to standard body surface area calculation method in the present field, dose used to human beings (calculated by 70 kg) should be above 112 mg/time with respect to the doses of rats. Obviously, as adopting normal clinic injection specification of 120 ml, the polydatin concentration in the solution should not be lower than 5.6 mg/ml.

The solvent adopted in the present invention can increase solubility of the polydatin in solutions evidently, and increase stability of the polydatin pharmaceutical composition in cold preservation, so the concentration of the polydatin can be adjusted not lower than 6 mg/ml to facilitate preparation of pharmaceutical compositions and clinic drugs. However, it is easily understood by technicians in the present field that, as applying the solvent according to the present invention, even if concentration of the polydatin is lower than 6 mg/ml, the solvent should be covered in the present invention.

In the polydatin pharmaceutical composition according to the present invention, the solvent -further comprise a predetermined amount of water.

Preferably, calculated by volume, the solvent comprises 50%˜80% alcohol, 0˜50% propanediol, and the remaining amount of water.

Preferably, the solvent is a mixed solvent containing a certain amount of propanediol, wherein calculated by volume, the solvent comprises 40%˜80% alcohol, 5%˜60% propanediol, and the remaining amount of water.

More preferably, calculated by volume, the solvent comprises 40%˜60% alcohol, 10%˜30% propanediol, and the remaining amount of water.

Most preferably, calculated by volume, the solvent comprises 45% alcohol, 20% propanediol, and the remaining amount of water.

In the polydatin pharmaceutical composition according to the present invention, the water of the mixed solvent can be purified water, water for injection, sodium chloride solution or glucose solution (for example, 0.9% sodium chloride water solution, 5% glucose solution, or glucose sodium chloride solution) acceptable in physiology, or buffer solution acceptable in physiology, for example, Na₂CO₃-NaHCO₃ buffer solution, Na₂HPO₄-NaH₂PO₄ buffer solution, Tris buffer solution and Hepes buffer solution. The polydatin pharmaceutical composition can select water from the above described species as applying in different administration ways, for example, as applying in injection administration, the water in the solvent is generally the water for injection, the normal saline, or the buffer solution, etc.; as applying in oral administration and etc., the purified water is adopted generally.

Preferably, the water in the mixed solvent is a buffer solution acceptable in physiology of pH6.0˜9.0, such as a Na₂HPO₄-NaH₂PO₄ buffer solution of pH about 6.0˜9.0.

More preferably, the water in the mixed solvent is preferred embodied as a buffer solution acceptable in physiology of pH7.5˜9.0.

Most preferably, the water in the mixed solvent is a buffer solution acceptable in physiology of pH8.5, such as a Na₂CO₃-NaHCO₃ buffer solution of pH8.5.

The polydatin pharmaceutical composition according to the present invention can be applied in parenteral injection administration. As applying in administration in vein, the polydatin pharmaceutical composition can adopt direct infusion administration or infusion administration after being diluted with 0.9% sodium chloride injection or 5% glucose injection; as applying in intramuscular administration, the polydatin pharmaceutical composition can adopt direct injection administration or injection administration after being diluted to a certain concentration with normal saline. The polydatin pharmaceutical composition according to the present invention can be applied as an oral administration solution, or further be made into a spray or an aerosol.

Accordingly, formulations of the polydatin pharmaceutical composition according to the present invention can be injection, oral solution, spray, or aerosol.

Further, the polydatin pharmaceutical composition according to the present invention comprises active component of unit dose, wherein the unit dose can comprise the polydatin of 3˜1500 mg, the polydatin of 6˜500 mg preferably, the polydatin of 50˜250 mg more preferably, and according to a preferable embodiment of the present invention, the polydatin of 100 mg. Volume of the solution containing the polydatin of unit dose can be 0.5˜250 ml, 2˜20 ml preferably, 2˜10 ml more preferably, and according to a preferable embodiment of the present invention, 5 ml.

The polydatin pharmaceutical composition according to the present invention can comprise other medicinal excipients used conventionally in medicine optionally. For example, the polydatin pharmaceutical composition according to the present invention can comprise optionally a material which increase viscosity of suspension, such as sodium carboxymethyl cellulose, sorbitol, methyl cellulose; can comprise optionally a proper stabilizer or antioxidant such as sodium bisulfite, or vitamin C; can comprise a proper taste agent to improve taste as applying in oral administration.

The present invention provides application of the pharmaceutical composition containing polydatin in preparing drugs which treat and prevent diseases relative to microcirculation disturbance. The polydatin pharmaceutical composition according to the present invention possesses evident effect in dilating microvessel, reducing blood viscosity in microvessel, inhibiting adhesion action of blood cells, so the polydatin drug composition possesses evident effect of improving microcirculation, can be applied to treat or prevent shock and diseases relative to microcirculation disturbance.

Pharmacodynamic experimental result indicates that comparing with a blank control group and a positive control group of dopamine, the polydatin injection has evident treating effect to dogs in hemorrhagic shock, increases evidently 24 h survival rate, 48 h survival rate and long-term survival rate of animals in shock, and has better effect to shock than the positive control drug of dopamine.

The present invention discovers a method of adopting mixed solvent to improve solubility of drugs through plenty of experiments and researches. Applying the method to medicine preparation can provide a solution containing polydatin of effective dose which is calculated according to prior research result. As a result, the present invention makes the pharmic effective component of polydatin be capable of being applied in clinic with effective dose, which has essential improvement and advancement comparing with prior art. Animal experiments and solution stability experiments indicate that the polydatin drug composition according to the present invention is safe and effective and has good stability, therefore has great prospect in clinic application.

These and other objectives, features, and advantages of the present invention will become apparent from the following detailed description, the accompanying drawings, and the appended claims.

### BRIEF DESCRIPTION OF THE DRAWINGS

FIG. 1 illustrates changes of average arterial pressure of dogs in hemorrhagic shock.

FIG. 2 illustrates comparison between change rates of pulse pressure difference of dogs in hemorrhagic shock.

Tables 1 through 11 are prescriptions for Embodiments 1 through 11 respectively.

Table 12 shows the effect of pH to solubility of polydatin.

Table 13 shows the solubility of polydatin in alcohol-water solution and normal saline.

Table 14 shows the solubility of polydatin in propanediol-water solution.

Table 15 shows the solubility of polydatin in alcohol-propanediol-water mixed solvent.

Table 16 shows the comparison between stability of polydatin pharmaceutical compositions prepared by different methods.

Table 17 shows another comparison between stability of polydatin pharmaceutical compositions prepared by different methods.

Table 18-1 shows the comparison between compatible stability of polydatin pharmaceutical compositions (diluted in 100 ml 0.9% NaCl injection).

Table 18-2 shows another comparison between compatible stability of polydatin pharmaceutical compositions (diluted in 250 ml 0.9% NaCl injection).

Table 19 shows the comparison between compatible stability of polydatin pharmaceutical compositions prepared with mixed solvents of different proportions.

Table 20 shows the comparison between survival times of dogs in hemorrhagic shock.

Table 21 shows the comparison between survival time of dogs in hemorrhagic shock and survival rate of different periods of time thereof (number/5).

### DETAILED DESCRIPTION OF THE PREFERRED EMBODIMENT

According to a first embodiment of the pharmaceutical composition of the present invention, a process of making polydatin aqueous solution for injection is provided.

According to a first exemplary illustration of the first embodiment, a process of making an aqueous solution for injection is described, wherein the aqueous solution has a composition comprising a quantity of polydatin, a quantity of NaOH aqueous solution at pH 8.5 and a quantity of 3.6% NaCl aqueous solution. According to the composition of the first exemplary illustration, the quantity of polydatin is 40 g, the quantity of NaOH solution is 8 L, and the quantity of 3.6% NaCl is used for adjusting the total volume of the composition into a 10 L solution, wherein the composition of the first exemplary illustration is divided into 1000 unit.

The process of making an aqueous solution for injection comprises the steps of dissolving 40 g polydatin in 8 L NaOH aqueous solution at pH 8.5; adding a quantity of NaCl until the total volume is 9.8 L; adjusting the pH to 8.5 and adding NaCl aqueous solution until the total volume is 10 L; filtering using 0.22 μm microfilter membrane; and dividing the solution into 1000 units and keeping in ampoules or bottles.

Embodiment A1-2: According to a second exemplary illustration of the first embodiment, a process of making an aqueous solution for injection is described, wherein the aqueous solution has a composition comprising a quantity of polydatin, a quantity of NaOH aqueous solution at pH 8.5, a quantity of propandiol, a quantity of Tween 80, and a quantity of 3.6% NaCl aqueous solution. According to the composition of the first exemplary illustration, the quantity of polydatin is 80 g, the quantity of NaOH solution is 6 L, the quantity of propandiol is 2 L, the quantity of Tween 80 is 50 ml, and the quantity of 3.6% NaCl is used for adjusting the total volume of the composition into a 10 L solution, wherein the composition of the first exemplary illustration is divided into 1000 unit.

The process of making an aqueous solution for injection in the embodiment A1-2 comprises the steps of dissolving 40 g polydatin in 2 L propandiol solution; adding 50 ml Tween 80 and NaOH aqueous solution respectively and mixing; adding a quantity of 3.6% NaCl until the total volume is 9.8 L; adjusting the pH to 8.5 and adding NaCl aqueous solution until the total volume is 10 L; filtering using 0.22 μm microfilter membrane; and dividing the solution into 1000 units and keeping in ampoules or bottles.

Embodiment A1-3: According to a third exemplary illustration of the first embodiment, a process of making an aqueous solution for injection is described, wherein the aqueous solution has a composition comprising a quantity of polydatin, a quantity of anhydrous alcohol, propandiol, and a quantity of sodium carbon. According to the composition of the third exemplary illustration, the quantity of polydatin is 100 g, the quantity of anhydrous alcohol is 2250 ml, the quantity of propandiol is 1000 ml, and the quantity of sodium carbonate is used for adjusting the total volume of the aqueous solution into 5000 ml. The aqueous solution in the embodiment A1-3 is diluted 25 to 50 times by 0.9% sodium fluoride or 5% glucose injection solution for administration.

The process of making an aqueous solution for injection in the embodiment A1-3 comprises the steps of dissolving 20 g polydatin in 450 ml anhydrous alcohol; adding 100 ml buffer solution and mixing by ultrasound or stirring for 2 to 5 minutes such that the polydatin is dissolved; adding 200 ml propandiol and the remaining buffer solution until the total volume is IL; filtering using 0.22 μm microfilter membrane; and refilling nitrogen and dividing the solution into 1000 units which are kept in brown ampoules or bottles. The buffer solution is prepared by mixing one unit of 0.1 mol/L sodium carbonate and nine units of 0.1 mol/L sodium hydrocarbonate.

Embodiment A2: A process of making a cool dry powder of polydatin for injection.

Embodiment A2-1: A process of making a polydatin solution is illustrated in embodiment A2-1. The process of making a polydatin solution in the embodiment A2-1 comprises the steps of dissolving 40 g polydatin in 8 L pH 8.5 NaOH aqueous solution; adding a quantity of 5% aqueous manitol solution until the total volume is 9.8 L; adjusting the pH to 8.5 and adding 3.6% NaCl aqueous solution until the total volume is 10 L; filtering using 0.22 μm microfilter membrane; and dividing the solution into 1000 units and keeping in vial for dry powder.

A process of freeze dry is also illustrated in the embodiment A2-1, comprising the step of obtaining the solution obtained from the process of making a polydatin solution, and placing the solution in a freeze dry condition with a temperature at −35° C. such that the solution is kept in −30° C. for 3 hours; vacuum pumping at condensation temperature at −40° C. and increasing the temperature to 40° C. gradually such that the frozen solution is increased gradually; drying the frozen solution and that the freeze dry product is obtained. The product can then be packed and pressed with piston, enclosed and labeled as a polydatin injection powder. The powder is adapted for dissolving in physiological saline for injection use.

Embodiment A2-2: A process of making a polydatin solution is illustrated in embodiment A2-2. The process of making a polydatin solution in the embodiment A2-2 comprises the steps of dissolving 80 g polydatin in 8 L pH 9.5 NaOH aqueous solution; adding a quantity of 5% aqueous manitol solution until the total volume is 9.8 L; adjusting the pH to 8.5 and adding 3.6% NaCl aqueous solution until the total volume is 10 L; filtering using 0.22 μm microfilter membrane; and dividing the solution into 1000 units and keeping in vial for freeze dry powder.

A process of freeze dry is also illustrated in the embodiment A2-2, comprising the step of obtaining the solution obtained from the process of making a polydatin solution, and placing the solution in a freeze dry condition with a temperature at −30° C. such that the solution is kept in −30° C. for 3 hours; vacuum pumping at condensation temperature at −40° C. and increasing the temperature to 40° C. gradually such that the frozen solution is increased gradually; drying the frozen solution and that the freeze dry product is obtained. The product can then be packed and pressed with piston, enclosed and labeled as a polydatin injection powder. The powder is adapted for dissolving in physiological saline for injection use.

A process of making an injection solution of the present invention is illustrated in the embodiment A2-2, comprising the steps of mixing 2 L propandiol and 500 ml 0.5% hydrochloric acid; adding a predetermined quantity of physiology saline such that the total volume is 10 L, filtering using 0.22 μm microfilter membrane; and dividing the solution into 1000 units and keeping in vial. Each of the vials comprises 10 ml of injection solution.

A package comprises a 10 ml injection solution and a unit of freeze dry powder is preferred such that the freeze dry powder is capable of dissolving in the injection solution for injection.

Embodiment A3: A process of making a composition of polydatin in tablet form. 1000 tablet each having a composition of 50 mg polydatin are prepared according to the routine making process, and that the composition of polydatin comprises a quantity of polydatin, a quantity of lactose, a quantity of starch, a quantity of polyethylene pyrrolidone K30, and a quantity of magnesium stearate. In embodiment A3, 50 g of polydatin, 107 g of lactose, 25 g of starch, 16 g of polyethylene pyrrolidone K30, and 2 g of magnesium stearate are used and that the total weight is 200 g.

The polydatin is mixed with lactose and starch to form a mixture. The mixture is transformed into a granule form by using polyethylene pyrrolidone K30 and passing through a No. 16 model unit. After drying, the granule is mixed with magnesium stearate and pressed into a predetermined shape. Other polydatin in tablet form having different active composition of polydatin can be made by varying the ratio of the quantity of polydatin and vehicle, or the force of pressing device.

Embodiment A4: A process of making a composition of polydatin in capsule form, comprising the steps of: mixing a quantity of polydatin, a quantity of lactose, a quantity of microcrystalized cellulose, and a quantity of magnesium stearate. In the embodiment A4, 200 capsules having 50 mg active composition of polydatin are prepared from 10 g polydatin, 19.5 g lactose, 10 g microcrystalized cellulose, and 0.5 g magnesium stearate, wherein a total weight is 40 g.

Embodiment A5: A process of making a composition of polydatin in ointment form, comprising the steps of: mixing and stirring a quantity of polydatin and a quantity of olive oil; and adding a quantity of dissolved white Vaseline while stirring; and mixing thoroughly. The ointment is then canned. In embodiment A5, a 1% polydatin ointment is prepared from 1 g polydatin, 10 g olive oil, and 89 g white Vaseline wherein the total weight is 100 g.

Embodiment A6: A process of making a composition of polydatin in suppository form, comprising the steps of: mixing and stirring a quantity of polydatin and a quantity of glycerol; and adding a quantity of dissolved glycerol gelatin while stirring; and mixing thoroughly. The suppository is then canned. In embodiment A5, a 1% polydatin suppository is prepared from 1 g polydatin, 5 g glycerol, and 84 g glycerol gelatin wherein the total weight is 100 g.

Embodiment A7: A treatment action for shock related to blood loss of a composition of polydatin.

The observation is based on the experiment carried out comprising the steps of
(1) anaesthetizing two groups, namely a treatment group and a control group, of SD rats with Urethane Ketamine, inducing bleeding by intubulation at femoral artery and recording blood pressure; wherein femoral vein is used for medication and blood transfusion and cremasteric specimen is prepared by Baez method;
(2) inducing bleeding from femoral artery such that an average arterial blood pressure is maintained between 5.1 to 5.6 kPa;
(3) administering a composition of polydatin injection through femoral vein after one hour wherein the dosage of the composition is 0.6 ml/kg, that the concentration of polydatin is 4 mg/ml and the dosage is 2.4 mg/kg in the treatment group and the composition of polydatin is replaced by physiological saline in the control group;
(4) conducting blood transfusion after 20 minutes such that the volume of blood loss due to bleeding is equal to the volume of blood loss due to blood transfusion; and
(5) observing for two hours and detaching the intubulation.

The records are made by using Olympus microscope, Hitachi monitoring system and physiological recording meter to observe and record microcirculation and blood dynamics. The survival period of each subject of the two groups are recorded.

### Result:

(1) Survival period: a ratio of the survival time of the treatment group and the control group is larger than 5.5, wherein 9/10 of the treatment group has a survival period of 24 hours, 8/10 of the treatment group has a survival period of 48 hours, and 6/10 of the treatment group has a survival period of 72 hours. All the subjects in the control group die within 24 hours.
(2) Blood pressure: After experienced shock for 1 hour, the average arterial blood pressure is 5.3+0.2 kPa. After 30 minutes of medication of polydatin, the arterial pressure is increased to 8.6+0.8 kPa in the treatment group, while there is no significant difference in blood pressure in the control group receiving physiological saline.
(3) Diameter of micro blood vessel: After experienced shock for 1 hour, the diameter of micro-aneurysm is reduced to 63.3% of the diameter in average before shock. The diameter is then increased by 20.3% after medication of polydatin and there is no significant difference of diameter in the control group.
(4) Number of opening capillaries: Before shock, the number of open capillary is 5.1±0.4 per μm². After induced bleeding, the number of open capillary is decreased to 2.2±0.6 per μm². The number increased to 4.2±0.5 per μm² in 10 minutes after polydatin medication. There is no significant difference of number of opening capillaries in the control group before and after the administration of physiological saline.

Embodiment A8: A treatment action for shock related to skin burn of a composition of polydatin.

The observation is based on the experiment carried out comprising the steps of: anaesthetizing two groups, namely a treatment group and a control group, of SD rats with Urethane Ketamine, burning about 35% of body surface below waist for 30 seconds with 80° C. water; administering 0.6 ml/kg polydatin injection (comprising 4 mg/ml polydatin and the dosage is 2.4 mg/kg) by venous injection after half hour of burning in the treatment group and replacing the polydatin injection by physiological saline in control group.

Result: The average survival period of the treatment group is longer than 13.3 hours while the average survival period of the control group is 7.0±2.6 hours.

The following pharmaceutical composition contains polydatin and solvent for dissolving the polydatin. Accordingly, the solvent comprises alcohol in a range between 40 and 95% by volume, propanediol in a range between 0 and 60% by volume, and water in remaining volume.

The pharmaceutical composition comprises polydatin of effective concentration, and is capable of keeping stable as being cold preserved. The composition is prepared to compatible solution according to clinic conventional usage and dose, and is capable of keeping the compatible solution stable and clarified.

Polydatin used in following experiments is provided by NEPTUNUS PHARMACEUTICAL TECHNOLOGY CENTER, has purity of 99.63%, batch number 20030829, whose particular preparation method refers to China patent application 200310112538.3.

Other experimental materials are products on sale without particular specification.

### Preparation of Polydatin Injection

Materials: anhydrous alcohol (or alcohol), analytically pure, Guangzhou No. 2 chemical reagent factory; water for injection, NEPTUNUS industry city; 0.9% sodium chloride solution, prepared with the water for injection; sodium chloride glucose solution, prepared with the water for injection; propanediol, analytically pure, Guangzhou No. 2 chemical reagent factory; carbonate buffer solution, prepared by weighing anhydrous sodium carbonate and sodium bicarbonate of proper amount and preparing with the water for injection, pH8.5˜9.0.

### EMBODIMENT 1

1. Prescription: as shown in Table 1.
2. Preparation method:

Measuring 3.50 L anhydrous alcohol, adding water for injection of proper amount, mixing, adding 400.00 g polydatin, dissolving with ultrasonic under 40° C., adding water for injection to scale, filtering with 0.2 μm microporous membrane, sealing in 1000 ampoule bottles, diluting with 0.9% sodium chloride injection or 5% glucose injection as about to use.

### EMBODIMENT 2

1. Prescription: as shown in Table 2.
2. Preparation method:

Measuring 2.50 L anhydrous alcohol, adding 0.9% sodium chloride solution of proper amount, mixing, adding 125.00 g polydatin, dissolving with ultrasonic under 40° C., adding sodium chloride solution to scale, filtering with 0.2 μm microporous membrane, sealing in 1000 ampoule bottles.

### EMBODIMENT 3

1. Prescription: as shown in Table 3.
2. Preparation method:

Measuring alcohol according to the prescription amount, adding 125.00 g polydatin, dissolving with ultrasonic under 40° C., adding alcohol to scale, filtering with 0.21 μm microporous membrane, sealing in 1000 ampoule bottles, diluting with 0.9% sodium chloride injection or 5% glucose injection as about to use.

### EMBODIMENT 4

1. Prescription: as shown in Table 4.
2. Preparation method:

Measuring 2.25 L anhydrous alcohol, adding 1.00 L propanediol and carbonate buffer solution, mixing, adding 100.00 g polydatin, dissolving with ultrasonic, adding buffer solution to scale, filtering with 0.45 μm microporous membrane, rough filtering. Filtering continuously with 0.22 μm microporous membrane, plate and frame ultrafilter, equipping pes membrane of 10000 molecular weight cut off, eliminating pyrogen. Sealing in brown volumetric flask with nitrogen.

### EMBODIMENT 5

1. Prescription: as shown in Table 5.

2. Preparation method:

Measuring anhydrous alcohol and propanediol according to the prescription amount, mixing, adding 325.00 g polydatin, dissolving with ultrasonic under 40° C., adding 0.9% sodium chloride solution to scale. Filtering with 0.45 μm microporous membrane, rough filtering. Filtering continuously with 0.22 μm microporous membrane, plate and frame ultrafilter, equipping pes membrane of 10000 molecular weight cut off, eliminating pyrogen. Sealing in brown volumetric flask with nitrogen. Diluting to proper concentration with 0.9% sodium chloride injection or 5% glucose injection as about to use.

### EMBODIMENT 6

1. Prescription: as shown in Table 6.
2. Preparation method:

Measuring alcohol and propanediol according to the prescription amount, mixing, adding 500.00 g polydatin, dissolving with ultrasonic under 40° C., adding water for injection to scale. Filtering with 0.45 μm microporous membrane, rough filtering. Filtering continuously with 0.22 μm microporous membrane, plate and frame ultrafilter, equipping pes membrane of 10000 molecular weight cut off, eliminating pyrogen. Sealing in brown volumetric flask with nitrogen. Diluting to proper concentration with 0.9% sodium chloride injection or 5% glucose injection as about to use.

### EMBODIMENT 7

1. Prescription: as shown in Table 7.
2. Preparation method:

Measuring alcohol, propanediol, and glucose sodium chloride solution according to the prescription amount, mixing, adding 150.00 g polydatin, dissolving with ultrasonic under 40° C., adding glucose sodium chloride solution to scale. Filtering with 0.45 μm microporous membrane, rough filtering. Filtering continuously with 0.22 μm microporous membrane, plate and frame ultrafilter, equipping pes membrane of 10000 molecular weight cut off, eliminating pyrogen. Sealing in brown volumetric flask with nitrogen.

Preparation of polydatin oral solution Materials: alcohol, analytically pure, Guangzhou No. 2 chemical reagent factory; propanediol, analytically pure, Guangzhou No. 2 chemical reagent factory; purified water, NEPTUNUS industry city.

### EMBODIMENT 8

1. Prescription: as shown in Table 8.
2. Preparation method:

Measuring alcohol, propanediol, and purified water according to the prescription amount, mixing, adding 110.00 g polydatin, dissolving with ultrasonic under 40° C., adding purified water to scale. Filtering with 0.45 μm microporous membrane, split charging to 1000 glass bottles.

### EMBODIMENT 9

1. Prescription: as shown in Table 9.
2. Preparation method:

Measuring alcohol, propanediol, and purified water according to the prescription amount, adding 120.00 g polydatin, dissolving with ultrasonic under 40° C., adding purified water to scale. Filtering with 0.45 μm microporous membrane, split charging.

### Preparation of Polydatin Spray

Materials: alcohol, analytically pure, Guangzhou No. 2 chemical reagent factory; propanediol, analytically pure, Guangzhou No. 2 chemical reagent factory; purified water, NEPTUNUS industry city.

### EMBODIMENT 10

1. Prescription: as shown in Table 10.
2. Preparation method:

Measuring alcohol, propanediol, and purified water according to the prescription amount mixing, adding 120.00 g polydatin, dissolving with ultrasonic under 40° C., adding purified water to scale. Filtering with 0.45 μm microporous membrane, rough filtering. Filtering with 0.22 μm microporous membrane, charging filtrate into ultrasonic atomizer. Most spray particles have diameters below 5 μm, can be absorbed into respiratory bronchus and pulmonary alveoli. Diluting to proper concentration with 0.9% sodium chloride injection or 5% glucose injection as about to use.

### Preparation of Polydatin Aerosol

### EMBODIMENT 11

1. Prescription: as shown in Table 11.
2. Preparation method:

Measuring 100.00 g polydatin, adding into mixed solvent of alcohol and propanediol, agitating to dissolve. Filtering with 0.45 μm microporous membrane, rough filtering. Filtering with 0.22 μm microporous membrane, charging filtrate by dose, sealing dose valve system, injecting F12 as increasing pressure, shaking up to obtain polydatin aerosol.

Comparison between solubility of polydatin in different solvents and mixed solvents

### EMBODIMENT 12

### Effect of pH to Solubility of Polydatin

1. Materials: Deionized water, NEPTUNUS industry city; sodium hydroxide, Dongguan dongjiang chemical reagent co., ltd.; hydrochloric acid, Dongguan dongjiang chemical reagent co., ltd.
2. Method: Regulating deionized water into pH of 5.0˜12.0 with 0.2N sodium hydroxide and 0.2N hydrochloric acid. Measuring precisely 20 ml each of above solutions, adding polydatin of proper amount respectively, treating with ultrasonic for 20 min under 40° C., supersaturating the solution, placing for 8 h under room temperature, filtering with 0.45 μm microporous membrane, wherein the filtrates are saturated solutions of polydatin in water solutions of different pHs. Adopting HPLC method to determine content of polydatin in the filtrates and calculate solubility of polydatin referring to document 20 (Wuye etc., determination content of resveratrol glycoside and tangerine peel glycoside in qing dan capsule with HPLC method, Chinese traditional patent medicine, 1999, 21(5): 226).
3. Result: Referring to Table 12, the result indicates that under room temperature, when pH of solvents ≦11.0, solubility of polydatin water solution is lower than mg/ml; when pH is 12, concentration of polydatin is about 5 mg/ml, but pH of final solution is about 10.

### EMBODIMENT 13

### Solubility of Polydatin in Alcohol-Water Solution and Normal Saline

1. Materials: Anhydrous alcohol, analytically pure, Guangzhou No.2 chemical reagent factory; purified water, NEPTUNUS industry city; 0.9% sodium chloride injection, batch number C040514-092, Sichuan kelun pharmaceutical co., ltd.
2. Method: Preparing alcohol water solutions having concentration of 15%, 5 30%, 40%, 50%, 60%, 70%, 80%, 90%, 95%(v/v) with anhydrous alcohol and purified water. Precisely Measuring water, anhydrous alcohol, 0.9% sodium chloride injection (normal saline) and 20 ml alcohol water solution of each above concentration, adding polydatin of proper amount respectively, treating with ultrasonic for 20 min under 40 □, supersaturating the solution, placing for 8 h under room temperature, filtering with 0.45 μm microporous membrane, wherein the filtrates are saturated solutions of polydatin in alcohol water solutions of different concentration. Adopting HPLC method to determine content of polydatin in the filtrates and calculate solubility of polydatin referring to document 20.
3. Result: Referring to Table 13, the result indicates that under room temperature, solubility of polydatin in purified water and normal saline is lower than 0.5 mg/ml, solubility in anhydrous alcohol is about 20 mg/ml, and the highest solubility is in an alcohol water solution of a certain concentration range, for example, solubility of polydatin is up to 80 mg/ml in 70% (v/v) alcohol water solution. Solubility of polydatin is higher than 20 mg/ml in 50%˜95% alcohol water solution, which is higher than solubility in anhydrous alcohol. The result indicates that 50%˜95% alcohol water solution has cosolvency effect to polydatin.

### EMBODIMENT 14

### Solubility of Polydatin in Propanediol-Water Solution

Materials: 1,2-propanediol, analytically pure, Guangzhou No. 2 chemical reagent factory; purified water, NEPTUNUS industry city.

Method: Preparing propanediol water solutions having concentration of at least 20%(v/v) with propanediol and purified water. Precisely measuring propanediol and 20 ml propanediol water solution of each above concentration, adding polydatin of proper amount respectively, treating with ultrasonic for 20 min under 40° C., supersaturating the solution, placing for 8 h under room temperature, filtering with 0.45 μm microporous membrane, wherein the filtrates are saturated solutions of polydatin in propanediol water solutions of different concentration. Adopting HPLC method to determine content of polydatin in the filtrates and calculate solubility of polydatin referring to document 20.

Result: Referring to Table 14, the result indicates that under room temperature, solubility of polydatin in propanediol water solution increases as concentration of propanediol in the solutions increases, and the highest solubility is about 60 mg/ml. In 20 % propanediol water solution, solubility of polydatin is about 3.5 mg/ml, and the solution is not stable, and precipitates solute of polydatin after placing for 12 h.

### EMBODIMENT 15

### Solubility of Polydatin in Alcohol-Propanediol-Water Mixed Solvent

1. Materials: Anhydrous alcohol, analytically pure, Guangzhou No. 2 chemical reagent factory; 1,2-propanediol, analytically pure, Guangzhou No. 2 chemical reagent factory; purified water, NEPTUNUS industry city.

2. Method: Precisely measuring alcohol water solution of 45%, 60%, 75%(v/v), precisely adding propanediol at volume proportions of 10:1, 10:2, 10:2.5, 10:3, 10:4, preparing mixed solvents. The mixed solvents obtained contain propanediol of about 10%˜30%(v/v), alcohol of about 32%˜68%(v/v). Precisely measuring 20 ml of each above mixed solvent, adding polydatin of proper amount respectively, treating with ultrasonic for 20 min under 40° C., supersaturating the solution, placing for 8 h under room temperature, filtering with 0.45 μm microporous membrane, wherein the filtrates are saturated solutions of polydatin in different mixed solutions. Adopting HPLC method to determine content of polydatin in the filtrates and calculate solubility of polydatin referring to document 20.

3. Result: Referring to Table 15, the result indicates that:

A. In mixed solvents prepared with 60% alcohol-water or 75% alcohol-water with propanediol within a certain proportion, solubility of polydatin is higher than the alcohol-water solution before mixing, and higher than the single solvent propanediol, in another word, as to alcohol-water and propanediol, mixed solvent of a certain proportion range has cosolvent effect to polydatin.

B. In view of general technical requirements (as adopting alcohol to be solvent for injection, the highest concentration is about 50%, (Zhao Xinxian is editor in chief, “Chinese traditional drugs injection”, Guangdong science and technical publishing house, 2000: p128). Preparing mixed solvent with 60% alcohol-water and propanediol in the proportion of 10:2.0˜10:4.0 preferably, wherein actual content of alcohol in the solution is no more than 50%, and concentration of polydatin is higher than 60 mg/ml.

### Study on Stability of Polydatin Drug Composition

The present invention further studies on stability of polydatin composition, observes stability of solution placed long in cold, and observes stability of solution which is diluted to effective concentration with 0.9% sodium chloride injection or 5% glucose injection. Experimental result indicates that polydatin drug composition has best stability as being prepared with 60% alcohol solution and propanediol in the proportion of about 10:2.5.

### EMBODIMENT 16

### Comparison Between Stability of Polydatin Drug Compositions Prepared Different Methods

1. Materials:

Sample 1: polydatin injection, prepared according to the Embodiment 3;

Sample 2: polydatin injection, prepared according to the Embodiment 4;

Sample 3: control injection, prepared according to Embodiment 1-1 in China patent application 02134928.2;

Sample 4: control injection, prepared according to Embodiment 1-2 in China patent application 02134928.2.

(injection prescriptions of the Embodiment 1-1 and the Embodiment 1-2 in the China patent application 02134928.2 are as below:

Embodiment 1-1: polydatin of 40 g; NaOH water solution of pH8.5, 8 L; diluting to 10 L with 3.6% NaCl water solution, and split charging to 1000 bottles.

Embodiment 1-2: polydatin of 80 g; NaOH water solution of pH8.5, 6 L; propanediol of 2 L; tween of 50 ml; diluting to 10 L with 3.6% NaCl water solution, and split charging to 1000 bottles

2. Method

Placing the above four samples in a refrigerator of 4° C. for 1 week, taking out the samples, observing whether there is solute precipitated; taking the above samples of proper amount (equivalent to polydatin of 100 mg), adding 0.9% NaCl injection of 100 ml, placing under room temperature for 3 hours, observing whether there is solute precipitated.

3. Result

Referring to Table 5, the result indicates that:

A. The sample 1 and the sample 2 can keep stability of solutions in environment of 4° C. for long-term storage, while the sample 3 and the sample 4 precipitate crystal of active component of polydatin in environment of 4° C. for long-term storage;

B. The sample 1 and the sample 2 can keep stability of solutions as being diluted in 0.9% NaCl injection in the proportion of 5:100 (v/v) and placed under room temperature for 3 hours, while the sample 3 and the sample 4 can not keep stability of solutions and precipitate active component as being diluted with the same condition.

EMBODIMENT 17

### Observation of Stability of Polydatin Drug Compositions Prepared by Different Methods in Cold Preservation

1. Materials:

Sample 1˜6: polydatin injections, prepared according to the Embodiment 13, concentrations of the alcohol-water solvent are 50%, 60%, 70%, 80%, 90%, 95% respectively;

Sample 7˜10: polydatin injections, prepared according to the Embodiment 14, concentrations of the propanediol-water solvent are 50%, 65%, 80%, 100% respectively;

Sample 11˜26: polydatin injections, prepared according to the Embodiment 15, the mixed solvents are prepared with 45%, 60%, 75% alcohol-water and propanediol in the proportion of 10:1.0˜10:4.0 respectively.

2. Method: Placing the above sample solutions in a refrigerator of 4° C. for 1 week, and observing whether there is solute precipitated.

3. Result: Referring to Table 17, the result indicates that, the sample solutions prepared with alcohol-water and mixed solvents are stable in cold preservation; while the sample solutions prepared with propanediol-water can only keep stable in condition of propanediol of high concentration (≧80%).

### EMBODIMENT 18

### Study on Compatible (Diluted) Stability of Polydatin Drug Compositions Prepared by Different Methods in Cold Preservation

1. Materials: According to the Embodiment 15, preparing mixed solvents with 45%, 60%, 75% alcohol-water and propanediol in proportion of 10/1, 10/2, 10/2.5, 10/3, 10/4, and preparing sample solutions respectively. Besides, according to the Embodiments 13, 14, preparing sample solutions with solvents of 40%˜95% alcohol-water and 50%˜100% propanediol-water.

2. Method: Taking the above sample solutions of proper amount (equivalent to polydatin of about 100 mg), adding into 100 ml and 250 ml 0.9% NaCl injection respectively, placing for 3 hours, observing whether there is solute precipitated. Placing the compatible solution having no solute precipitated into a refrigerator of 4° C. and observing continuously.

3. Result: Referring to Tables 18-1 and 18-2, the result indicates that:

A. The sample solutions prepared with alcohol-water or water-alcohol-propanediol mixed solvents has stable, clarified compatible solution after diluting with 100 ml and 250 ml 0.9% NaCl injection (normal saline); while the sample solutions prepared with propanediol-water solution of different concentrations precipitate a great deal of precipitation after compatibility, and the compatible solutions are not stable;

B. Compatible stability of the mixed solutions is better than single solvent of alcohol-water as observing the compatible solutions in cold preservation (4° C.)

C. The sample solutions have different compatible stability when the mixed solutions have different preparation proportions, wherein the mixed solutions prepared with 60%, 75% alcohol-water have better compatible stability.

The result indicates that polydatin injections prepared with alcohol-water solution or water-alcohol-propanediol mixed solution of a certain proportion range can meet general clinic requirements, further, concentration of alcohol in mixed solution is preferably embodied as ≧60%.

### EMBODIMENT 19

### Study on Compatible Stability of Polydatin Drug Compositions Prepared by Mixed Solvents

1. Materials: Polydatin injections: preparing mixed solvents with 60% alcohol-water and propanediol in the proportions of 10:2.0, 10:2.5, 10:3.0, 10:4.0, preparing 5 ml sample solutions having unit measurement containing 100 mg polydatin.

2. Method: Diluting the above sample solutions into 100 ml 0.9% NaCl injection, and observing stability of the compatible solutions.

3. Result: Referring to Table 19, the result indicates that, the polydatin injection which is prepared with mixed solvent prepared with 60% alcohol-water and propanediol in the proportion of 10:2.5 (v/v) has best compatible stability. In view of general technical requirements (as adopting alcohol to be solvent for injection, the highest concentration is about 50%, (Zhao Xinxian is editor in chief, “Chinese traditional drugs injection”, Guangdong science and technical publishing house, 2000: pl28), the polydatin injection prepared according to the proportion has best clinic application value.

Pharmacodynamic study on polydatin drug composition Embodiment 20 treatment effect of polydatin drug compositions to dogs in hemorrhagic shock

1. Materials

A. Tested materials

Polydatin injection: preparing according to the embodiment 4.

Normal saline for injection: produced by Anhui huayuan biological pharmacy co., ltd., batch number 2003021203.

B. Control materials

Dopamine injection: Guangzhou mingxin pharmaceuticals co., ltd., batch number MC3105

Experimental animal

Beagle dogs: provided by the first military medical university, certification number: 001321, permission number of animal: SYXK (Yue) 2003-0007, female or male is not limited, body weight of 8˜12 kg.

2. Experimental method

A. Anaesthesia and operation

Anaesthetising the animals by intravenous injection with 3% sodium pentobarbital of 1.0 ml/kg after weighing. After anaesthetising, fixing the animals in a supine position, treating with tracheal intubation. Separating bilateral femoral artery, and unilateral femoral vein, treating with vascular intubation. Arterial intubation is used to record blood pressure and bloodletting, and venous intubation is used to administrate and reinfuse liquid.

B. Preparation of model of dogs in hemorrhagic shock

Observing and recording normal blood pressure and respiratory frequency of the animals before bloodletting, and recording heart rate accurately by recording electrocardiogram. Turning on switch communicating a unilateral femoral artery tube and a blank transfusion bottle to make blood flow naturally into a disinfected transfusion bottle containing heparin normal saline, making average arterial pressure decrease to 40 mmHg within 15 min, and making the average arterial pressure maintain in the level for 120 min by adjusting blood volume released from the transfusion bottle (calculated by that average whole blood volume of dogs is 94.1 ml/kg, the blood loss takes about 60% of the whole blood volume). Then administration treating by groups respectively, finishing intravenous infusion administration within 120 min, observing various indexes for 120 min, pulling out all the tubes, sealing skin, and recording survival time of the animals (observing the survival time for 7 days at most, the animals surviving more than 7 days are treated as long-term survival, whose survival time is calculated as 168 hours). Making sure that the operation and experimental process are in aseptic operation, and paying attention to keep body temperature of the animals at 37° C. during the experimental process.

C. Administration dose and method

Administration dose: administration doses of polydatin injections of three dose groups are 1.8, 3.0, 5.0 mg/kg respectively, and administration dose of the control group of dopamine is 3.0 mg/kg.

Administration method: diluting the tested drugs in normal saline having volume of ⅓ of the blood loss, treating with continuous intravenous infusion administration for 120 min.

D. Observation indexes

The experiment observes arterial blood pressure, heart rate and respiratory frequency, and observes the survival time of the animals and survival rate of different periods of time thereof. Observation time of the indexes is normal, 0, 60, and 120 min after shock, 30, 60, 90 and 120 min in administration process and 0, 30, 60, 90, and 120 min after administration finishes.

3. Experimental result

A. Effect of polydatin injection to blood pressure of dogs in hemorrhagic shock.

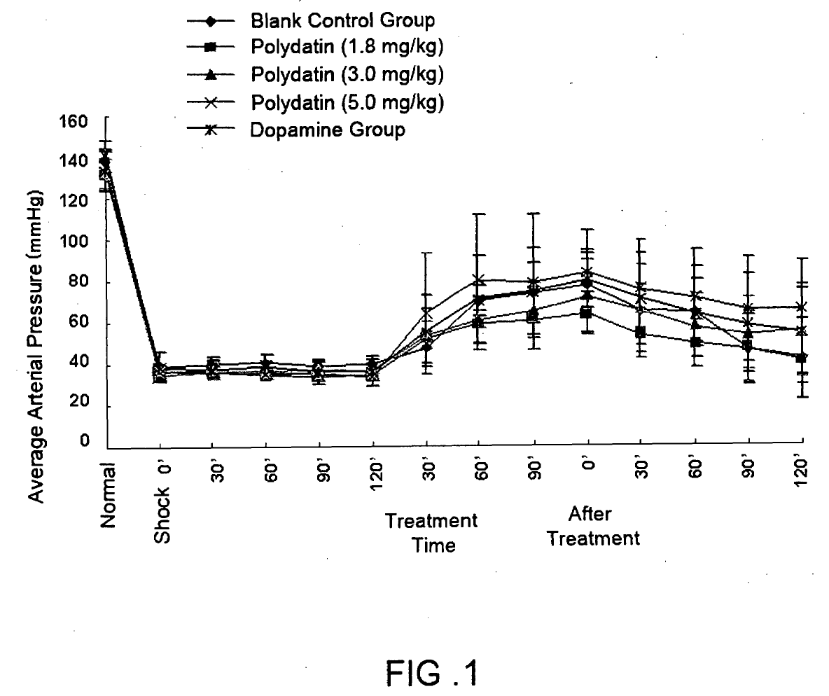

Referring to FIG. 1, the result indicates that, comparing with the blank control group and the treatment group of dopamine, treating with polydatin of high dose (5.0 mg/kg) can maintain blood pressure of dogs after acute hemorrhage, and the blood pressure recovers fast after treatment starts, range increases, and maintains at a high level after 2 hours the treatment finishes.

B. Effect of polydatin injection to pulse pressure difference of dogs in hemorrhagic shock

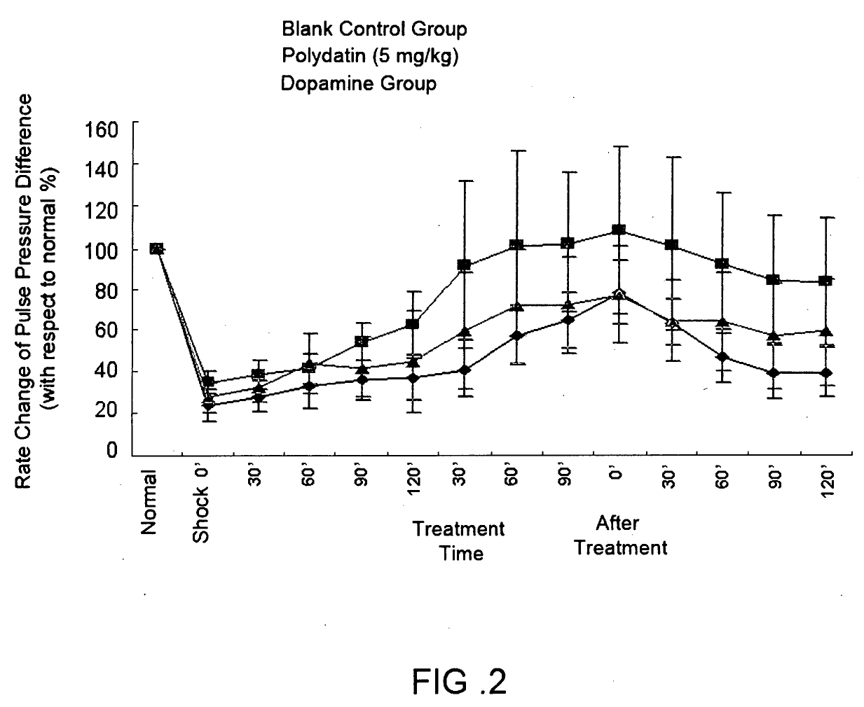

Referring to FIG. 2, the result indicates that, treating the dogs in hemorrhagic shock with polydatin injection can recover partially decreased pulse pressure difference because of acute hemorrhage, make pulse pressure recover to normal level, and maintain at a high level after stopping back transfusion. Changing trend of pulse pressure difference of the blank control group and the treatment group of dopamine is as same as the polydatin groups, but increasing range is obviously lower than the polydatin groups.

C. Changing of survival time and survival rate of dogs in hemorrhagic shock of different groups

Referring to Table 20 and Table 21, the result indicates that, comparing with the blank control group, survival time of the polydatin groups of different doses is extended, and survival rate of different periods of time is increased; in 5 experimental animals of each group, only 1 animal survives in the blank control group, while 3 animals survive in the treatment group of polydatin of 1.8 mg/kg, the other two dose groups have 4 animals survives respectively; and the treatment group of dopamine has same survival number of animals as the polydatin group of lowest dose.

4. Experimental conclusion

The above pharmacodynamic experiment result indicates that, comparing with the blank control group and the positive control group of dopamine, polydatin injections have obvious treatment effect to dogs in hemorrhagic shock, and increase evidently 24 hours, 48 hours and long-term survival rate of animals after shock, and have better treatment effect to shock than the positive control drug of dopamine.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

TABLE 1
(Prescription for Embodiment 1)

| material | dose | |
|---|---|---|
| polydatin | 400.00 | g |
| anhydrous alcohol | 3.50 | L |
| water for injection | add to 5.00 | L |
| product | 1000 | bottles |

TABLE 2
(Prescription for Embodiment 2)

| material | dose | |
|---|---|---|
| polydatin | 125.00 | g |
| anhydrous alcohol | 2.50 | L |
| 0.9% sodium chloride solution | add to 5.00 | L |
| product | 1000 | bottles |

TABLE 3
(Prescription for Embodiment 3)

| material | dose | |
|---|---|---|
| polydatin | 125.00 | g |
| alcohol | add to 5.00 | L |
| product | 1000 | bottles |

TABLE 4
(Prescription for Embodiment 4)

| material | dose | |
|---|---|---|
| polydatin | 100.00 | g |
| propanediol | 1.00 | L |
| anhydrous alcohol | 2.25 | L |
| Na₂CO₃—NaHCO₃ buffer solution | add to 5.00 | L |
| product | 1000 | bottles |

TABLE 5
(Prescription for Embodiment 5)

| material | dose | |
|---|---|---|
| polydatin | 325.00 | g |
| propanediol | 3.00 | L |
| anhydrous alcohol | 2.00 | L |
| 0.9% sodium chloride solution | add to 5.00 | L |
| product | 1000 | bottles |

TABLE 6
(Prescription for Embodiment 6)

| material | dose | |
|---|---|---|
| polydatin | 500.00 | g |
| propanediol | 0.50 | L |
| alcohol | 3.68 | L |
| water for injection | add to 5.00 | L |
| product | 1000 | bottles |

TABLE 7
(Prescription for Embodiment 7)

| material | dose | |
|---|---|---|
| polydatin | 150.00 | g |
| propanediol | 1.25 | L |
| alcohol | 10.50 | L |
| glucose sodium chloride solution | add to 25.00 | L |
| product | 100 | bottles |

TABLE 8
(Prescription for Embodiment 8)

| material | dose | |
|---|---|---|
| polydatin | 110.00 | g |
| propanediol | 0.18 | L |
| alcohol | 1.16 | L |
| purified water | add to 2.00 | L |
| split charging | 1000 | bottles |

TABLE 9
(Prescription for Embodiment 9)

| material | dose | |
|---|---|---|
| polydatin | 120.00 | g |
| alcohol | 1.68 | L |
| propanediol | 0.10 | L |
| purified water | add to 2.00 | L |
| split charging | 1000 | bottles |

TABLE 10
(Prescription for Embodiment 10)

| material | dose | |
|---|---|---|
| polydatin | 120.00 | g |
| alcohol | 8.42 | L |
| propanediol | 1.00 | L |
| purified water | add to 20.00 | L |
| split charging | 1000 | bottles |

TABLE 11
(Prescription for Embodiment 11)

| material | dose | |
|---|---|---|
| polydatin | 100.00 | g |
| propanediol | 1.00 | L |
| alcohol | 2.25 | L |
| freon F12 | add to 5000.00 | g |
| split charging | 1000 | bottles |

TABLE 12
(Effect of pH to solubility of polydatin)

| pH of solvent | solubility (mg/ml) |
|---|---|
| 5.0 | <0.5 |
| 6.0 | <0.5 |
| 7.0 | <0.5 |
| 8.0 | <0.5 |
| 9.0 | <0.5 |
| 10.0 | <0.5 |
| 11.0 | 0.7 |
| 12.0 | 4.5 |

TABLE 13
(solubility of polydatin in alcohol-water
solution and normal saline)

| content of alcohol (%) | solubility (mg/ml) |
|---|---|
| 0 | <0.5 |
| 15 | 1.1 |
| 30 | 6.2 |
| 40 | 9.2 |
| 50 | 26.1 |
| 60 | 42.4 |
| 70 | 80.9 |
| 80 | 76.8 |
| 90 | 75.4 |
| 95 | 30.7 |
| 100 | 21.4 |
| normal saline | <0.5 |

TABLE 14
(solubility of polydatin in propanediol-water solution)

| content of propanediol (%) | solubility (mg/ml) |
|---|---|
| 20 | 3.5 |
| 35 | 4.9 |
| 50 | 24.4 |
| 65 | 28.3 |
| 80 | 33.6 |
| 100 | 60.2 |

TABLE 15
(solubility of polydatin in alcohol-propanediol-water
mixed solvent mg/ml)

| | alcohol water | | |
|---|---|---|---|
| alcohol water/propanediol | 45% | 60% | 75% |
| 10/—* | 20.2 | 42.5 | 75.1 |
| 10/1 | 27.8 | 55.7 | 81.5 |
| 10/2 | 34.3 | 66.4 | 89.8 |
| 10/2.5 | 39.5 | 75.3 | 92.9 |
| 10/3 | 42.4 | 93.3 | 98.4 |
| 10/4 | 52.2 | 95.2 | 116.1 |

Annotation:
a. *no propanediol;
b. solubility of polydatin in propanediol is about 60 mg/ml

TABLE 16
(comparison between stability of polydatin pharmaceutical
compositions prepared by different methods)

| | sample | sample | sample | sample |
|---|---|---|---|---|
| placing condition | 1 | 2 | 3 | 4 |
| original solution under 4° C. | − | − | + | + |
| for a week | | | | |
| diluting in 0.9% NaCl injection | − | − | + | + |
| under room temperature for 3 | | | | |
| hours | | | | |

Annotation:
−: no crystal precipitated;
+: crystal precipitated

TABLE 17
(comparison between stability of polydatin pharmaceutical
compositions prepared by different methods)

| | sample | sample 7, | sample 9, | sample |
|---|---|---|---|---|
| placing condition | 1~6 | 8 | 10 | 11~26 |
| under 4° C. for a week | − | + | − | + |

Annotation:
−: no crystal precipitated;
+: crystal precipitated

TABLE 18-1
comparison between compatible stability of polydatin pharmaceutical
compositions (diluted in 100 ml 0.9% NaCl injection)

| | alcohol water | | |
|---|---|---|---|
| alcohol water/propanediol | 45% | 60% | 75% |
| 10/1 | − | − | − |
| 10/2 | − | − | − |
| 10/2.5 | − | − | − |
| 10/3 | − | − | − |
| 10/4 | −* | − | − |

| | |
|---|---|
| alcohol-water | −# |
| propanediol-water | + |

TABLE 18-2
comparison between compatible stability of polydatin pharmaceutical
compositions (diluted in 250 ml 0.9% NaCl injection)

| | alcohol water | | |
|---|---|---|---|
| alcohol water/propanediol | 45% | 60% | 75% |
| 10/1 | − | − | − |
| 10/2 | − | − | − |
| 10/2.5 | − | − | − |
| 10/3 | − | − | − |
| 10/4 | − | − | − |

| | |
|---|---|
| alcohol-water | −# |
| propanediol-water | + |

Annotation:
−: no crystal precipitated;
+: crystal precipitated;
*little crystal precipitated after placing for 3~6 hours;
#little crystal precipitated in cold preservation (4° C.)

TABLE 19
comparison between compatible stability of polydatin
pharmaceutical compositions prepared with mixed
solvents of different proportions

| | proportion of mixed solvents | | | |
|---|---|---|---|---|
| | 10:2.0 | 10:2.5 | 10:3.0 | 10:4.0 |
| stability of | ≧2 weeks | ≧4 weeks | ≧2 weeks | ~1 week |
| compatible solution | | | | |

TABLE 20
comparison between survival times of dogs in hemorrhagic shock

| group | | average survival time (hour, x ± s) |
|---|---|---|
| blank control group | | 38.90 ± 72.20 |
| polydatin injection | 1.8 mg/kg | 103.10 ± 88.87 |
| groups | 3.0 mg/kg | 135.40 ± 72.90 |
| | 5.0 mg/kg | 135.40 ± 72.90 |
| dopamine group | 3.0 mg/kg | 103.00 ± 89.01 |

Annotation:
survival time of animals of long-term survival (surviving for more than 7 days) is calculated with 168 hours.

TABLE 21
comparison between survival time of dogs in hemorrhagic shock and
survival rate of different periods of time thereof (number/5)

| | | 24 | 48 | 72 | 168 |
|---|---|---|---|---|---|
| group | | hours | hours | hours | hours |
| blank control group | | 1 | 1 | 1 | 1 |
| polydatin | 1.8 mg/kg | 3 | 3 | 3 | 3 |
| injection groups | 3.0 mg/kg | 4 | 4 | 4 | 4 |
| | 5.0 mg/kg | 4 | 4 | 4 | 4 |
| dopamine group | 3.0 mg/kg | 3 | 3 | 3 | 3 |

What is claimed is:

1. A composition, comprising polydatin and solvent for dissolving said polydatin, wherein said solvent comprises alcohol in a range between 40 and 95% by volume, propanediol in a range between 0 and 60% by volume, and water in remaining volume.

2. The composition, as recited in claim 1, wherein said polydatin in said solvent has a concentration in a range between 6 and 150 mg/ml.

3. The composition, as recited in claim 1, wherein said polydatin in said solvent has a concentration in a range between 20 and 100 mg/ml.

4. The composition, as recited in claim 1, wherein said alcohol has a range between 50 and 80% by volume, said propanediol has a range between 0 and 50% by volume, and the remaining volume is water.

5. The composition, as recited in claim 1, wherein said alcohol has a range between 40 and 80% by volume, said propanediol has a range between 5 and 60% by volume, and the remaining volume is water.

6. The composition, as recited in claim 1, wherein said alcohol has a range between 40 and 60% by volume, said propanediol has a range between 10 and 30% by volume, and the remaining volume is water.

7. The composition, as recited in claim 1, wherein a volume ratio among said alcohol, said propanediol, and water is 45:20:35.

8. The composition, as recited in claim 1, wherein said water includes at least one solution selected from the group consisting of purified water, water for injection, normal saline, glucose solution, glucose sodium chloride solution, and buffer solution which has pH in a range between 6.0 and 9.0.

9. The composition, as recited in claim 1, which is in a pharmaceutically acceptable administrative form selected from the group consisting of an intravenous injection, and intramuscular injection, an oral administration, a spray solution, and an aerosol form.

10. A method for treating and preventing microcirculation obstruction related diseases including hemorrhagic shock, comprising a step of:
administrating a pharmaceutically acceptable composition, wherein said composition comprises polydatin and solvent for dissolving said polydatin, wherein said solvent comprises alcohol in a range between 40 and 95% by volume, propanediol in a range between 0 and 60% by volume, and water in remaining volume.

11. The method, as recited in claim 10, wherein said composition is in a pharmaceutically acceptable administrative form selected from the group consisting of an intravenous injection, and intramuscular injection, an oral administration, a spray solution, and an aerosol form.

12. The method, as recited in claim 11, wherein a volume ratio among said alcohol, said propanediol, and water is 45:20:35.

13. The method, as recited in claim 12, wherein said water includes at least one solution selected from the group consisting of purified water, water for injection, normal saline, glucose solution, glucose sodium chloride solution, and buffer solution.

14. The method, as recited in claim 13, wherein said polydatin in said solvent has a concentration in a range between 6 and 150 mg/ml.

15. The method, as recited in claim 13, wherein said polydatin in said solvent has a concentration in a range between 20 and 100 mg/ml.

16. A method of manufacturing a composition, comprising the steps of:
(a) providing a predetermined amount of polydatin;
(b) adding a solvent for dissolving said polydatin, wherein said solvent comprises alcohol in a range between 40 and 95% by volume, propanediol in a range between 0 and 60% by volume, and water in remaining volume.

17. The method, as recited in claim 16, wherein a volume ratio among said alcohol, said propanediol, and water is 45:20:35.

18. The method, as recited in claim 17, wherein said water includes at least one solution selected from the group consisting of purified water, water for injection, normal saline, glucose solution, glucose sodium chloride solution, and buffer solution.

19. The method, as recited in claim 18, wherein said polydatin in said solvent has a concentration in a range between 6 and 150 mg/ml.

20. The method, as recited in claim 18, wherein said polydatin in said solvent has a concentration in a range between 20 and 100 mg/ml.
